(19) United States
(12) Patent Application Publication
(10) Pub. No.: US 2010/0094097 A1
(43) Pub. Date: Apr. 15, 2010

(54) SYSTEM AND METHOD FOR TAKING RESPONSIVE ACTION TO HUMAN BIOSIGNALS

(76) Inventors: Charles Liu, Chapel Hill, NC (US); L. Scott Bloebaum, Cary, NC (US)

Correspondence Address: WARREN A. SKLAR (SOER);RENNER, OTTO, BOISSELLE & SKLAR, LLP, 1621 EUCLID AVENUE, 19TH FLOOR, CLEVELAND, OH, 44115, US

(21) Appl. No.: 12/251,910

(22) Filed: Oct. 15, 2008

(51) Int. Cl. A61B 5/00

(52) U.S. Cl. 600/300

(57) ABSTRACT

To enhance interaction and use with electronic devices, the present disclosure describes several improved systems and methods of taking responsive action to a detected human mental state. A training signal containing biosignal data corresponding to a mental state of the user while the user concentrates on a perceptual cue may be established. In some embodiments, user input or other information may be stored in association with the training signal. Biosignal data from the user may be monitored at an appropriate time and, if a match between the monitored biosignal data and the training signal is determined, a predetermined response is taken by an electronic device.

### TECHNICAL FIELD OF THE INVENTION

The technology of the present disclosure relates generally to human-machine interfaces and, more particularly, to a system and method for taking a responsive action in accordance with human biosignals.

### BACKGROUND

The term “biosignals” refers to various signals that are detectable from a person. Prominent biosignals are electrical signals produced by the heart, muscles and brain. Signals from the heart may be monitored by electrocardiogram (EKG or ECG), signals from the muscles may be monitored by electromyogram (EMG), and signals from the brain may be monitored by electroencephalogram (EEG). Biosignals have been studied for the treatment of medical conditions.

More recently, there has been interest in studying biosignals for use in human emotion recognition and human-computer interactions for the purpose of treating persons with disabilities. There also have been attempts to extend the use of biosignals into consumer areas. For example, Emotiv Systems of 600 Townsend Street—East Wing, Penthouse, San Francisco, Calif. 94103 markets a wearable headset for use while playing video games. The headset includes several EEG sensors. An associated software development kit (SDK) allows game developers to produce games that react to a player's emotional state while the player uses conventional control inputs. NeuroSky, Inc. of 226 Airport Parkway #638, San Jose, Calif., 95110 markets a similar wearable headset and SDK. The goal of these products is to capture biosignals, and interpret the biosignals to recognize a person's mental and/or emotional state. These systems rely on training to establish recognizable patterns.

### SUMMARY

To enhance interaction and use with electronic devices, the present disclosure describes several improved systems and methods of taking responsive action to a detected human mental state. As used herein, the term mental state expressly includes emotional state. Through the descriptions herein, a number of specific exemplary situations in which the disclosed systems and methods may be used are described. It will be appreciated that the disclosed systems and methods may be used in a wide variety of situations other than these specific examples.

In one embodiment, the user trains an electronic device while concentrating on a visual cue to establish a training signal indicative of the user's mental state. The user also associates an action with the training signal. During a subsequent use operation, if the electronic device matches a detected mental state with the training signal, the electronic device undertakes the associated action. Exemplary pairs of visual cues and actions include a corporate logo and a search for a nearest retail location. Another action may be to determine directions to the nearest retail location that matches the corporate logo. For example, the logo may be for the user's favorite pizza restaurant and, upon establishing a match, the electronic device may place a call to the restaurant so that the user may speak with an employee of the restaurant to place a take out order.

In another embodiment, the user trains an electronic device while concentrating on a visual cue to establish a training signal indicative of the user's mental state. Later, during a use operation, the user may think of the cue or look at objects that might match the cue. When the mental state of the user matches the training signal, the user may be alerted to the match condition. This may be useful when the user sees an object of interest and would like to distinguish a matching object from plural objects at a later point in time. For instance, if the user sees a handbag (e.g., a purse) belonging to another person and may want to purchase the same or similar handbag later, the user may establish the training signal while observing the purse. Later, while shopping in a store or through the Internet, the user may be presented with a large variety of handbags but cannot determine which one is the same as or closely matches the handbag that was originally observed. While the user is shopping, the electronic device may be used to monitor the user's mental state for a match to the training signal and, if a match occurs, the user may be alerted to the match. The alert may indicate that the currently observed handbag from the other handbags observed during the shopping experience may be the same as or very similar to the originally observed handbag.

In another embodiment, a search string may be created by matching a mental state to a previously stored training signal that has been associated with text. Additional text may be incorporated in the search string by converting words spoken at the time of conducting the match into text.

In another embodiment, the user may be reminded of directions to a location by matching mental state while driving to training signals that were established in advance. For instance, each training signal may be associated with a landmark and when the user sees the landmark while driving, a match may be made. Further, a directional prompt that was associated with the matched training signal may be presented to the user.

Through the description that follows, additional specific examples of taking an action in response to detected human biosignals will be described. It will be appreciated that there are additional and alternative operational scenarios in which the disclosed systems and methods may be used. Accordingly, the presentation of specific examples is for purposes of explanation only. Thus, the presentation of specific examples is not intended to be limiting of the subject matter recited in the appended claims.

According to one aspect of the disclosure, a method of taking action in response to biosignals detected from a user includes establishing a training signal containing biosignal data corresponding to a mental state of the user while the user concentrates on a perceptual cue; associating a user specified action to be carried out by an electronic device with the training signal; monitoring biosignal data from the user; and comparing the monitored biosignal data to the training signal and, if a match between the monitored biosignal data and the training signal is determined, commanding the electronic device to carry out the user specified action.

According to one embodiment of the method, the perceptual cue includes a visual representation of an item that is physically observed by the user at the time of establishing the training signal.

According to one embodiment of the method, the perceptual cue includes a trademark.

According to one embodiment of the method, the user specified action relates to one of a calling function, a messaging function, an audiovisual content playback function, a search function, or a navigation function.

According to one embodiment of the method, the perceptual cue includes a visual cue associated with a location, and a position of the electronic device is determined at the time of establishing the training signal.

According to one embodiment of the method, the user specified action is determining return directions to the position.

According to one embodiment of the method, the user specified action is automatically carried out if the match is made with a level of confidence that is above a predetermined threshold.

According to one embodiment of the method, the user specified action is established by recording a macro of steps.

According to one embodiment of the method, establishing the training signal includes establishing an ordered set of training signals, each containing biosignal data corresponding to a mental state of the user while the user concentrates on a perceptual cue; for each training signal, the associated user specified action is outputting a navigational prompt associated with the training signal; the monitoring of biosignal data is carried out as the user travels to a destination; and if a match between the monitored biosignal data and one of the training signals is determined, the commanding of the electronic device includes outputting the corresponding navigational prompt to the user.

According to one embodiment of the method, at least one of the perceptual cues is a representation of a landmark at which a navigational prompt is desired.

According to one embodiment of the method, at least one of the perceptual cues is a trademark associated with a landmark at which a navigational prompt is desired.

According to one embodiment of the method, the method further includes detecting turns made by the user and advancing through the ordered set of training signals based on the detected turns.

According to one embodiment of the method, the user specified action is constructing a search string using text representing search criteria that is associated with the training signal.

According to one embodiment of the method, the method further includes receiving voice input from the user.

According to one embodiment of the method, the method further includes converting the voice input to text.

According to one embodiment of the method, the converted text is made part of the search string.

According to one embodiment of the method, the voice input is received during the monitoring.

According to another aspect of the disclosure, a system for taking action in response to biosignals detected from a user includes a biosignal detection headset configured to detect biosignals from a user that are indicative of a mental state of the user and output corresponding biosignal data; and an electronic device that includes an interface to receive the biosignal data from the biosignal detection headset and a control circuit that is configured to establish a training signal containing biosignal data corresponding to a mental state of the user while the user concentrates on a perceptual cue; associate a user specified action to be carried out by the electronic device with the training signal; monitor biosignal data from the user; and compare the monitored biosignal data to the training signal and, if a match between the monitored biosignal data and the training signal is determined, command the electronic device to carry out the user specified action.

According to one embodiment of the system, the control circuit establishes an ordered set of training signals, each containing biosignal data corresponding to a mental state of the user while the user concentrates on a perceptual cue; for each training signal, the associated user specified action is outputting a navigational prompt associated with the training signal; biosignal data is monitored as the user travels to a destination; and if a match between the monitored biosignal data and one of the training signals is determined, the corresponding navigational prompt is output to the user.

According to one embodiment of the system, the user specified action is constructing a search string using text representing search criteria that is associated with the training signal.

These and further features will be apparent with reference to the following description and attached drawings. In the description and drawings, particular embodiments of the invention have been disclosed in detail as being indicative of some of the ways in which the principles of the invention may be employed, but it is understood that the invention is not limited correspondingly in scope. Rather, the invention includes all changes, modifications and equivalents coming within the scope of the claims appended hereto.

Features that are described and/or illustrated with respect to one embodiment may be used in the same way or in a similar way in one or more other embodiments and/or in combination with or instead of the features of the other embodiments.

### BRIEF DESCRIPTION OF THE DRAWINGS

FIG. 1 is a schematic view of an exemplary system for taking responsive action to human biosignals; and

FIGS. 2-5 are flow charts representing exemplary methods of taking responsive action to human biosignals using the system of FIG. 1.

### DETAILED DESCRIPTION OF EMBODIMENTS

Embodiments will now be described with reference to the drawings, wherein like reference numerals are used to refer to like elements throughout. It will be understood that the figures are not necessarily to scale.

In the present document, embodiments are described primarily in the context of a portable radio communications device, such as the illustrated mobile telephone. It will be appreciated, however, that the exemplary context of a mobile telephone is not the only operational environment in which aspects of the disclosed systems and methods may be used. Therefore, the techniques described in this document may be applied to any type of appropriate electronic device, a primary example of which is a computer, such as a laptop computer or a desktop computer. But other examples include, without limitation, a media player, a gaming device, an electronic organizer, a personal digital assistant (PDA), etc.

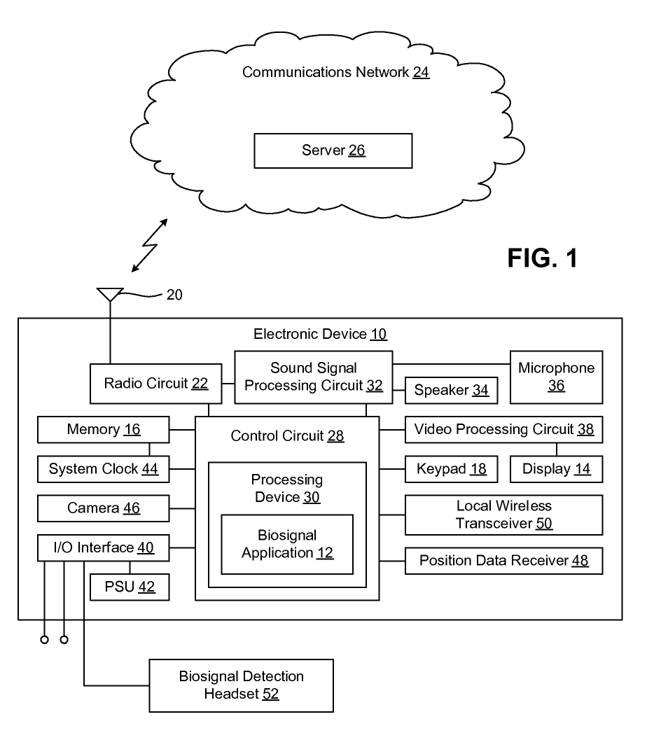

Referring initially to FIG. 1, a system for taking responsive action to human biosignals includes an electronic device 10. The electronic device 10 includes a biosignal application 12 that is configured to acquire training signals that represent sample mental states of a user for subsequent matching to future mental states, monitoring those future mental states and performing the matching, and carryout an appropriate responsive action when a match is made. Additional details and operation of the biosignal application 12 will be described in greater detail below. The biosignal application 12 may be embodied as executable code that is resident in and executed by the electronic device 10. In one embodiment, the biosignal application 12 may be one or more programs that are stored on a computer or machine readable medium. The biosignal application 12 may be a stand-alone software application or form a part of a software application that carries out additional tasks related to the electronic device 10.

Also, through the following description, exemplary techniques for taking action in response to detected biosignals are described. It will be appreciated that through the description of the exemplary techniques, a description of steps that may be carried out in part by executing software is described. The described steps are the foundation from which a programmer of ordinary skill in the art may write code to implement the described functionality. As such, a computer program listing is omitted for the sake of brevity. However, the described steps may be considered a method that the corresponding device is configured to carry out. Also, while the biosignal application 12 is implemented in software in accordance with an embodiment, such functionality could also be carried out via dedicated hardware or firmware, or some combination of hardware, firmware and/or software.

The electronic device of the illustrated embodiment is a mobile telephone, but will be referred to as the electronic device 10. As indicated, the electronic device 10 may be a device other than a mobile telephone.

The electronic device 10 may include a display 14. The display 14 displays information to a user such as operating state, time, telephone numbers, contact information, various menus, etc., that enable the user to utilize the various features of the electronic device 10. The display 14 also may be used to visually display content received by the electronic device 10 and/or retrieved from a memory 16 of the electronic device 10. The display 14 may be used to present images, video and other graphics to the user, such as photographs, mobile television content, Internet pages, and video associated with games.

A keypad 18 provides for a variety of user input operations. For example, the keypad 18 may include alphanumeric keys for allowing entry of alphanumeric information (e.g., telephone numbers, phone lists, contact information, notes, text, etc.), special function keys (e.g., a call send and answer key, multimedia playback control keys, a camera shutter button, etc.), navigation and select keys or a pointing device, and so forth. Keys or key-like functionality also may be embodied as a touch screen associated with the display 18. Also, the display 14 and keypad 18 may be used in conjunction with one another to implement soft key functionality.

The electronic device 10 includes communications circuitry that enables the electronic device 10 to establish a communications with another device. Communications may include calls, data transfers, and the like. Calls may take any suitable form such as, but not limited to, voice calls and video calls. The calls may be carried out over a cellular circuit-switched network or may be in the form of a voice over Internet Protocol (VoIP) call that is established over a packet-switched capability of a cellular network or over an alternative packet-switched network (e.g., a network compatible with IEEE 802.11, which is commonly referred to as WiFi, or a network compatible with IEEE 802.16, which is commonly referred to as WiMAX), for example. Data transfers may include, but are not limited to, receiving streaming content (e.g., streaming audio, streaming video, etc.), receiving data feeds (e.g., pushed data, podcasts, really simple syndication (RSS) data feeds data feeds), downloading and/or uploading data (e.g., image files, video files, audio files, ring tones, Internet content, etc.), receiving or sending messages (e.g., text messages, instant messages, electronic mail messages, multimedia messages), and so forth. This data may be processed by the electronic device 10, including storing the data in the memory 16, executing applications to allow user interaction with the data, displaying video and/or image content associated with the data, outputting audio sounds associated with the data, and so forth.

In the exemplary embodiment, the communications circuitry may include an antenna 20 coupled to a radio circuit 22. The radio circuit 22 includes a radio frequency transmitter and receiver for transmitting and receiving signals via the antenna 20. The radio circuit 22 may be configured to operate in a mobile communications system. Radio circuit 22 types for interaction with a mobile radio network and/or broadcasting network include, but are not limited to, global system for mobile communications (GSM), code division multiple access (CDMA), wideband CDMA (WCDMA), general packet radio service (GPRS), WiFi, WiMAX, digital video broadcasting-handheld (DVB-H), integrated services digital broadcasting (ISDB), high speed packet access (HSPA), etc., as well as advanced versions of these standards or any other appropriate standard. It will be appreciated that the electronic device 10 may be capable of communicating using more than one standard. Therefore, the antenna 20 and the radio circuit 22 may represent one or more than one radio transceiver.

The communications system may include a communications network 24 having a server 26 (or servers) for managing calls placed by and destined to the electronic device 10, transmitting data to and receiving data from the electronic device 10 and carrying out any other support functions. The server 26 communicates with the electronic device 10 via a transmission medium. The transmission medium may be any appropriate device or assembly, including, for example, a communications base station (e.g., a cellular service tower, or “cell” tower), a wireless access point, a satellite, etc. The network 24 may support the communications activity of multiple electronic devices 10 and other types of end user devices. As will be appreciated, the server 26 may be configured as a typical computer system used to carry out server functions and may include a processor configured to execute software containing logical instructions that embody the functions of the server 26 and a memory to store such software and any related databases. In alternative arrangements, the electronic device 10 may wirelessly communicate directly with another electronic device 10 (e.g., another mobile telephone or a computer) and without an intervening network.

The electronic device 10 may include a primary control circuit 28 that is configured to carry out overall control of the functions and operations of the electronic device 10. The control circuit 28 may include a processing device 30, such as a central processing unit (CPU), microcontroller or microprocessor. The processing device 30 executes code stored in a memory (not shown) within the control circuit 28 and/or in a separate memory, such as the memory 16, in order to carry out operation of the electronic device 10. For instance, the processing device 30 may execute and the memory 16 may store code that implements the biosignal application 12. The memory 16 may be, for example, one or more of a buffer, a flash memory, a hard drive, a removable media, a volatile memory, a non-volatile memory, a random access memory (RAM), or other suitable device. In a typical arrangement, the memory 16 may include a non-volatile memory for long term data storage and a volatile memory that functions as system memory for the control circuit 28. The memory 16 may exchange data with the control circuit 28 over a data bus. Accompanying control lines and an address bus between the memory 16 and the control circuit 28 also may be present.

The electronic device 10 further includes a sound signal processing circuit 32 for processing audio signals transmitted by and received from the radio circuit 22. Coupled to the sound processing circuit 32 are a speaker 30 for and a microphone 36 that enable a user to listen and speak via the electronic device 10. The radio circuit 22 and sound processing circuit 32 are each coupled to the control circuit 28 so as to carry out overall operation. Audio data may be passed from the control circuit 28 to the sound signal processing circuit 32 for playback to the user. The audio data may include, for example, audio data from an audio file stored by the memory 16 and retrieved by the control circuit 28, or received audio data such as in the form of voice communications or streaming audio data from a mobile radio service. The sound processing circuit 32 may include any appropriate buffers, decoders, amplifiers and so forth.

The display 14 may be coupled to the control circuit 28 by a video processing circuit 38 that converts video data to a video signal used to drive the display 14. The video processing circuit 38 may include any appropriate buffers, decoders, video data processors and so forth. The video data may be generated by the control circuit 28, retrieved from a video file that is stored in the memory 16, derived from an incoming video data stream that is received by the radio circuit 22 or obtained by any other suitable method.

The electronic device 10 may further include one or more input/output (I/O) interface(s) 40. The I/O interface(s) 40 may be in the form of typical mobile telephone I/O interfaces and may include one or more electrical connectors. The I/O interfaces 40 may form one or more data ports for connecting the electronic device 10 to another device (e.g., a computer) or an accessory (e.g., a personal handsfree (PHF) device) via a cable. Further, operating power may be received over the I/O interface(s) 40 and power to charge a battery of a power supply unit (PSU) 42 within the electronic device 10 may be received over the I/O interface(s) 40. The PSU 42 may supply power to operate the electronic device 10 in the absence of an external power source.

The electronic device 10 also may include various other components. For instance, a system clock 44 may clock components such as the control circuit 28 and the memory 16. A camera 46 may be present for taking digital pictures and/or movies. Image and/or video files corresponding to the pictures and/or movies may be stored in the memory 16. A position data receiver 48, such as a global positioning system (GPS) receiver, Galileo satellite system receiver or the like, may be involved in determining the location of the electronic device 10. A local wireless transceiver 50, such as an infrared transceiver and/or an RF transceiver (e.g., a Bluetooth chipset) may be used to establish communication with a nearby device, such as an accessory (e.g., a PHF device), another mobile radio terminal, a computer or another device.

Various exemplary functions involving the use of biosignals will now be described. Many of the functions may have particular relevance to the users of portable devices, such as the exemplary mobile telephone. However, some of the functions also may be used in connection with more stationary electronic devices. Each of the described functions involves capturing biosignals, interpreting the biosignals and recognizing commonalities between compared biosignals. While equipment for detecting biosignals and techniques for interpreting and processing biosignals are in their infancy in terms of technological development, the general approach to using the signals is understood. Therefore, the principles relied upon for the detection, interpretation and recognition of patterns among biosignals will not be described in great detail in this document.

To detect biosignals, the electronic device may be operatively interfaced with a biosignal detection headset 52. The biosignal detection headset 52 may be a commercially available headset for the detection of biosignals from the brain or head of the user. Biosignals data captured with the biosignal detection headset 52 may be indicative of mental state of the user.

In the illustrated embodiment, the biosignal detection headset 52 is connected to the electronic device 10 through a wired connection with one of the I/O interfaces 40 of the electronic device 10. In other embodiments, the biosignal detection headset 52 may include a wireless transceiver for communicating with the electronic device 10 through the local wireless transceiver 50 using a wireless interface. Also, while the processing to carry out the described functions is conducted by the electronic device 10 in the illustrated examples, at least some of the processing may be carried out by the server 26. For example, raw biosignals may be transmitted to the server 26 for processing, and commands, changes in state variable, and other data resulting from the processing of the raw biosignals may be transmitted back to the electronic device 10.

Some of the exemplary methods described in this document are described in connection with concentrating on a visual cue. It will be appreciated that other types of cues may be used, such as an auditory cue, a scent, or an abstract idea or thought of the user. Also, cues that result from a combination senses and/or mental impressions may be used. Therefore, cues originating from the senses, memories and/or thoughts may be referred generally as perceptual cues.

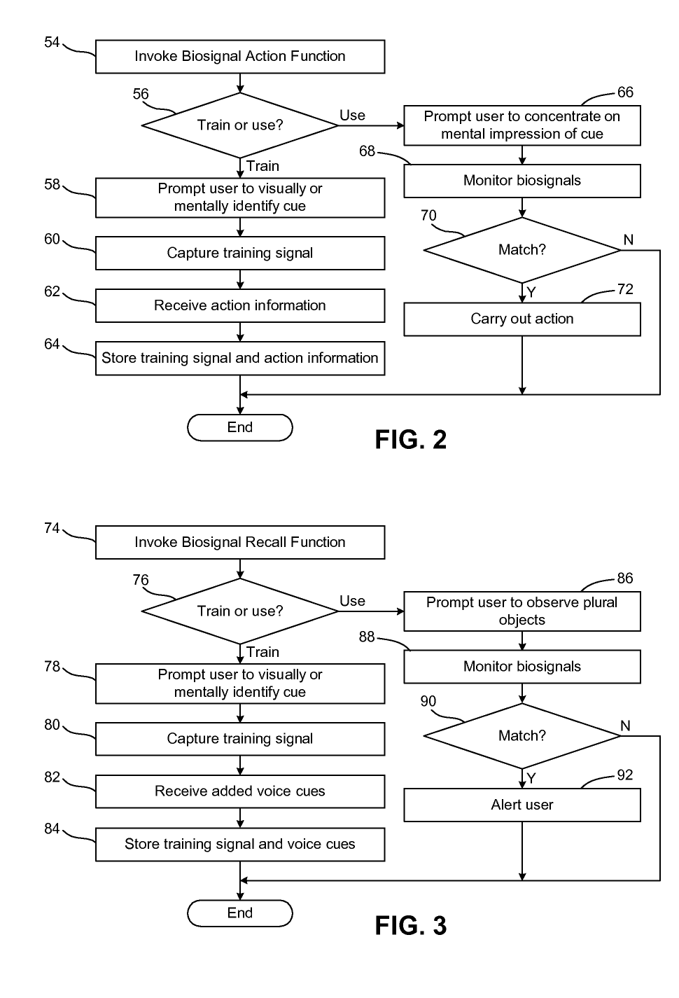

With additional reference to FIG. 2, illustrated are logical operations to implement an exemplary method of taking responsive action to human biosignals. The exemplary method may be carried out by executing an embodiment of the biosignal application 12, for example. Thus, the flow chart of FIG. 2 may be thought of as depicting steps of a method carried out by the electronic device 10. Although FIG. 2 shows a specific order of executing functional logic blocks, the order of executing the blocks may be changed relative to the order shown. Also, two or more blocks shown in succession may be executed concurrently or with partial concurrence. Certain blocks also may be omitted.

In the exemplary method represented by FIG. 2, a specified action is taken when the mental state of the user matches a previous mental state as determined when the user concentrated on a visual cue. This method may be referred to as a biosignal action function. The method represented by FIG. 2, establishes associations between biosignals and a particular visual cue (or, more generally, a particular perceptual cue) and then uses the established associations to take a specified action when the user thinks of the visual cue.

In one embodiment, the visual cue may be associated with a corporation, a store, a place, a person, an object, a pet, or other item or concept. In one described example, the visual cue is a corporate trademark (e.g., logo or brand name). And, in one specifically described example, the cue is a company logo associated with a major coffee house chain. The user may be prompted to concentrate on an image of the logo while a biosignal pattern is captured. Then, the user may establish an action to take and associate the action with the biosignal pattern that is associated with the brand represented by the logo. Subsequently, the user may concentrate on an actual image of the logo or the user's mental impression of the logo while biosignals are monitored from the user. If the monitored biosignal matches the previously captured in biosignal pattern, the action may be carried out. In this example, the action may be determining directions to the nearest retail coffee house associated with the logo. Another example may be to prepare a message with a take-out order for transmission to the nearest retail coffee house associated with the logo.

Of course, other uses for the method represented by FIG. 2 are possible. For instance, a biosignal pattern for a person may be associated with an action to dial a telephone number for the person.

Another example may involve establishing a biosignal pattern for a visual cue relating to a building, a landmark, a sign, a character written on a sign, or other memorable item that is located at a particular place (e.g., a train station in a city that is unfamiliar to the user and where signs may be written in an unfamiliar language). A position of the electronic device 10 may be determined at the time that the biosignal is captured. Later, the user may think of the mental impression that the user has for the visual cue and the electronic device 10 may generate return directions to the position or take some other action.

The logical flow for the biosignal action function may begin in block 54 where the biosignal application may be launched and the user may select the biosignal action function. Then, in block 56, the user may select a training mode or a use mode. If the user selects the training mode, the logical flow may proceed to block 58. In block 58 the biosignal application 12 may prompt the user to mentally concentrate on (e.g., think about) a cue. The user may concentrate on the cue by looking at an image or object that represents the cue. Following the example of a corporate logo as a cue, the user may look at the logo as it appears on a sign at a retail location. Alternatively, the user may look at an image of the logo, such as an image displayed on the display 14 of the electronic device 10. In other situations, the user may concentrate on a mental impression of the cue. That is, the user may think of what the cue looks like, but a physical representation of the cue may not be observed. The user may indicate to the electronic device 10 that the user is concentrating on the cue. This may be accomplished, for example, by pressing and holding a specified button from the keypad 18.

In block 60, while the user concentrates on the cue, the biosignal application 12 may capture a training signal (also known as a training vector). The training signal may contain biosignal data from the user that has a correlation to the visual cue and, hence, may contain a representation of the mental state of the user while the user thinks about the cue. Without intending to be bound by theory, it is believed that the user may repeatedly generate biosignal data that may be matched or correlated to the training signal when the user subsequently undertakes concentrated thinking about the visual cue, whether or not the user is physically observing the visual cue. In one embodiment, the capture of biosignal data may continue until sufficient data for a reliable training signal is captured. At that time, the user may be informed that the training is complete and may discontinue concentrating on the visual cue.

Next, in block 62, the user may associate an action with the visual cue. Several exemplary actions are described above. It will be understood that the action may be any action that the electronic device 10 is capable of performing in response to a later matching of the training signal with biosignal data that is monitored by the biosignal application 12. Therefore, in the exemplary context of a mobile telephone, the actions may relate to a calling function, a messaging function, an audiovisual content playback function, an Internet search function, and so forth. In one embodiment, the action may be selected from a menu of previously established actions. Also, the user may be provided with a mechanism to specify the action. For instance, the user may record a macro of the steps to be taken out by the electronic device.

In block 64, the training signal and the action may be stored. In one embodiment, the memory 16 may store a database in association with the biosignal application 12. The database may be used to store information used by the biosignal application 12, including training signals that form a repository of mental states corresponding to the visual cues for which the user may want to take an action. It is contemplated that different visual cues may invoke distinguishable mental states by the user. Therefore, the user may carry out the training routine more than once to store training signals for plural visual cues. Following the example of a logo for a coffee house, the user also may store a training signal for the visual cue of a logo for a pizza restaurant, may store another training signal for the visual cue of a logo for a bakery, and so forth.

In one embodiment, the user may be prompted to enter a text string label or other title for the training signal that is stored in block 64. The labeling may be used for management of training signals. The user may browse a directory of the labeled training signals to delete undesired training signals, revise the biosignal data stored as the training signal, and other operations.

As indicated, a position of the electronic device at the time that the training signal was captured also may be stored in association with the training signal.

In still another embodiment, the user may take a photograph of the visual cue or store an image of the visual cue. The photograph or image may be viewed at a later time to facilitating a match to the associated training signal or for presentation to the user after a match is made.

Returning to block 56, if the user chooses the use mode, the logical flow may proceed to block 66. In block 66, the user may be prompted to concentrate on his or her mental impression of a visual cue of interest. It is assumed that the user will have previously trained the electronic device 10 to store a training signal for the same visual cue. In most circumstances, the user may not have a physical representation of the visual cue to look at in the use mode. Therefore, the concentrating on the visual cue may rely on the user's recollection and mental impression of the visual cue. But there may be other circumstances when the user does have a physical representation of the visual cue to look at in the use mode. The visual cue upon which the user concentrates should be the same as a visual cue for which a training signal has been stored.

In block 68, while the user concentrates on the visual cue, the biosignal application 12 may monitor biosignal data from the biosignal detection headset 52. In block 70, the biosignal application 12 determines if the biosignal data contains a representation of the user's mental state that matches the user's mental state as represented in one of the training signals. In one embodiment, a simple data or signal matching engine may be employed. In other embodiments, analysis of the monitored biosignal data and the biosignal data in the training signals may be made to detect patterns or nuances in the respective datasets that match one another. The search for a match in block 70 may continue until the biosignal application 12 has sufficiently high confidence that a match is made or until a predetermined maximum capture time has been exceeded. If a match is not made, the user may repeat the attempt to make a match or the logical routine may end.

If a positive determination is made in block 70, the logical flow may proceed to block 72. In block 72, the biosignal application 12 may command the electronic device 10 to carry out the action that is stored in association with the matched training signal. In one embodiment, the action may be automatically carried out by default programming of the biosignal application 12 or by user specification that the action is to be automatically carried out. In another embodiment, following a match to a training signal in block 70, the user may be prompted to confirm that the action should be carried out. In another embodiment, if the match of block 70 is made with a level of confidence that is above a predetermined threshold, the action may be automatically carried out. In this embodiment, if the match is made with less than the predetermined threshold level of confidence, then the user may be prompted to confirm that the action should be carried out or may be given the opportunity to repeat the attempt to make a match.

Also, if a match is made, the label given to the matching training signal may be display to the user. If two or more possible matches are determined, the label of each potentially matching training signal may be displayed and the user may be provided with an option to select the intended match. The action associated with a selected match may then be carried out.

With additional reference to FIG. 3, illustrated are logical operations to implement an exemplary method of assisting a user recall a previous observation. The exemplary method may be carried out by executing an embodiment of the biosignal application 12, for example. Thus, the flow chart of FIG. 3 may be thought of as depicting steps of a method carried out by the electronic device 10. Although FIG. 3 shows a specific order of executing functional logic blocks, the order of executing the blocks may be changed relative to the order shown. Also, two or more blocks shown in succession may be executed concurrently or with partial concurrence. Certain blocks also may be omitted.

Some of the steps of the method represented by FIG. 3 are similar to or the same as steps found in the method represented by FIG. 2. Therefore, for the sake of brevity, similar or common steps will not be repeated in detail. Also, features and steps found in the method represented by FIG. 2 may be added to or replace features and steps found in the method represented by FIG. 3, and vice versa.

In the exemplary method represented by FIG. 3, an alert may be generated when the mental state of the user matches a previous mental state as determined when the user concentrated on a visual cue. This method may be referred to as a biosignal recall function. The method represented by FIG. 3, establishes associations between biosignals and a particular visual cue (or, more generally, a particular perceptual cue) and then uses the established associations to alert the user when there is a likely match in mental state because the user is likely thinking about or physically perceiving a matching visual cue. A level of confidence in the match also may be generated.

In one embodiment, the visual cue may be associated with a corporation, a store, a place, a person, an object, a pet, or other item or concept. In one described example, the visual cue is an object of interest. For instance, a person may see another person carrying a handbag and would like to be able to identify the same or similar handbag at a later time. In this case, the user may establish the training signal while directly observing the object. At a second point in time, such as when the user is shopping for handbag, the user may use the electronic device 10 to attempt to identify a handbag that invokes a correlating mental state as is represented by the training signal.

In other situations, the user may observe the cue at one point in time, then establish the training signal at a second point in time, and then attempt to identify the cue again at a third point in time. One example of this situation is a user that is working with law enforcement to identify a suspect alleged to be involved in a crime. In that case, the user may have observed the suspect and then subsequently established the training signal while thinking about the suspect. Then, at a third point in time, the user may be shown suspects (e.g., as part of a “line-up” or from a collection of images of persons) that meet the general description of the suspect in terms of height, weight, skin color, gender, etc. If one of the shown suspects invokes a correlating mental state as is represented by the training signal, then an alert of the match may be generated. In this situation, it may be desirable that only the law enforcement officer is informed of the match and not the user so as to avoid biasing the user, especially if the confidence level in the match is relatively low.

In another embodiment, the user may observe a person at one point in time and, either at that time or at a later time, establish a training signal while thinking about the person. The user also may associate information about the person, such as a name, contact information, a picture, etc., with the training signal. The user may want to recall this information and may do so by thinking about the person. If a match is made, the associated information may be displayed to the user. Also, there may be an instance where the user sees the person at some time after establishing the training signal, but cannot recall the person's name. In that situation, the user may attempt to match his or her mental state with the established training signal. If a match is made, the user may be alerted to the match and/or the stored information about the person may be recalled (e.g., displayed).

The logical flow for the biosignal recall function may begin in block 74 where the biosignal application may be launched and the user may select the biosignal recall function. Then, in block 76, the user may select a training mode or a use mode. If the user selects the training mode, the logical flow may proceed to block 78. In block 78 the biosignal application 12 may prompt the user to mentally concentrate on (e.g., think about) a cue. As indicated, the user may concentrate on the cue by looking at an image or object that represents the cue or the user may concentrate on an established mental impression of the cue. The user may indicate to the electronic device 10 that the user is concentrating on the cue. This may be accomplished, for example, by pressing and holding a specified button from the keypad 18.

In block 80, while the user concentrates on the cue, a biosignal application 12 may capture a training signal (also known as a training vector). The training signal may contain biosignal data from the user that has a correlation to the visual cue and, hence, may contain a representation of the mental state of the user while the user thinks about the cue. Without intending to be bound by theory, it is believed that the user may repeatedly generate biosignal data that may be matched or correlated to the training signal when the user subsequently undertakes concentrated thinking about the visual cue, whether or not the user is physically observing the visual cue. In one embodiment, the capture of biosignal data may continue until sufficient data for a reliable training signal is captured. At that time, the user may be informed that the training is complete and may discontinue concentrating on the visual cue. In the example of concentrating on a handbag, the user may try to observe and/or memorize as many characteristics as possible, such as style, size, color, logos, embellishments, features, etc. Also, the user may attempt to establish a training signal for the overall impression of the object and/or may attempt to establish separate training signals for each characteristic.

Next, in block 82, the user may associate additional information with the training signal, such as spoken words or phrases. Recordings of the spoken words or phrases may be played back during a use phase to assist the user in recalling characteristics of the visual cue and return the user to the user's mental state at the time of training.

In block 84, the training signal and any other added information may be stored, such as in the above-described database. Other exemplary information that may be stored with the training signal include a position of the electronic device at the time that the training signal was captured and a photograph of the visual cue for later viewing.

The user may carry out the training routine more than once to store training signals for plural visual cues. Following the example of a handbag, the user also may store a training signal for the visual cue of a pair of shoes, may store another training signal for the visual cue of a shirt, and so forth.

In one embodiment, the user may be prompted to enter a text string label or other title for the training signal that is stored in block 84. The labeling may be used for management of training signals. The user may browse a directory of the labeled training signals to delete undesired training signals, revise the biosignal data stored as the training signal, and other operations.

Returning to block 76, if the user chooses the use mode, the logical flow may proceed to block 86. In block 86, the user may be prompted observe various visual cues. For instance, following the example of shopping for a handbag that is the same as or similar to the previously observed handbag, the user may be in a store and looking through multiple handbags that are for sale or the user may browsing handbags shown on a website.

In one embodiment, using a label, an image, or voice cue, the user may choose a specific training signal that he or she is attempting to match. In this manner, the matching algorithm may narrow the scope of the matching between currently monitored biosignal data and previously stored training signals.

In block 88, while the user concentrates on the plural objects (or visual cues) that may possibly match the visual cue of interest and for which a training signal is previously stored, the biosignal application 12 may monitor biosignal data from the biosignal detection headset 52.

In block 90, the biosignal application 12 determines if the biosignal data contains a representation of the user's mental state that matches the user's mental state as represented in one of the training signals. In one embodiment, a simple data or signal matching engine may be employed. In other embodiments, analysis of the monitored biosignal data and the biosignal data in the training signals may be made to detect patterns or nuances in the respective datasets that match one another. The search for a match in block 90 may continue until the biosignal application 12 has sufficiently high confidence that a match is made or until a predetermined maximum capture time has been exceeded. If a match is not made, the user may repeat the attempt to make a match or the logical routine may end.

If a positive determination is made in block 90, the logical flow may proceed to block 92. In block 92, the biosignal application 12 may alert the user that a match to the training signal has been made. In one embodiment, a level of confidence in the match also may be displayed. For instance, if the matching logic is eighty percent confident that a match has occurred, a message may be displayed stating that a possible match with eighty percent confidence is made. The label for the training signal also may be displayed. In one embodiment, an auditory alert may be used to inform the user of the match.

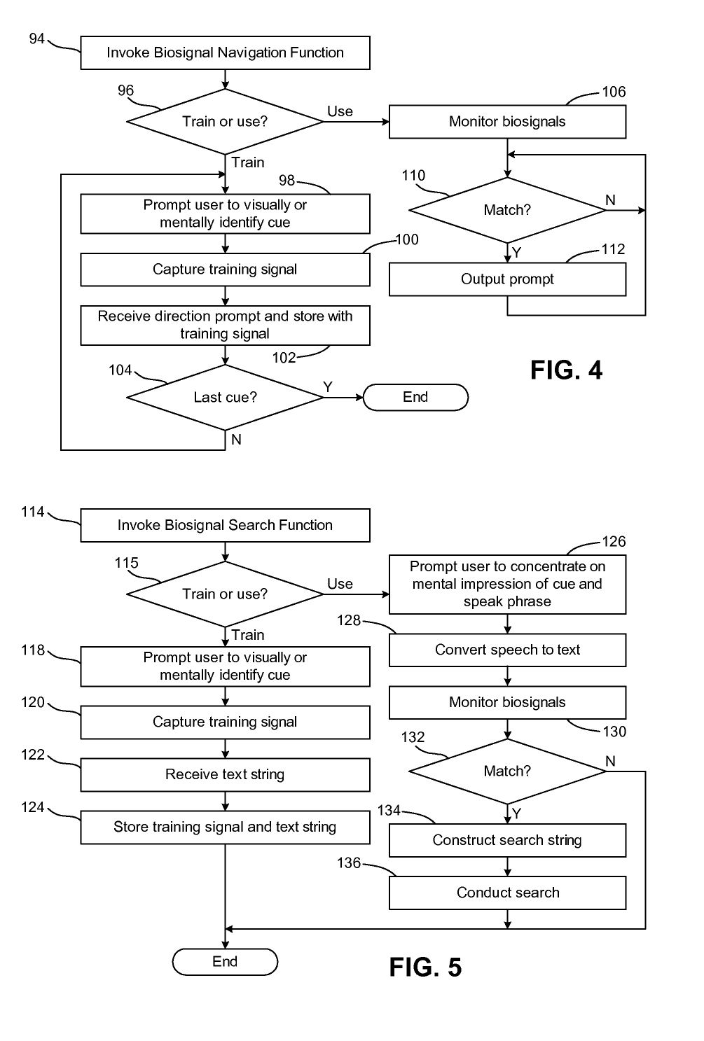

With additional reference to FIG. 4, illustrated are logical operations to implement an exemplary method of navigating to a desired destination. The exemplary method may be carried out by executing an embodiment of the biosignal application 12, for example. Thus, the flow chart of FIG. 4 may be thought of as depicting steps of a method carried out by the electronic device 10. Although FIG. 4 shows a specific order of executing functional logic blocks, the order of executing the blocks may be changed relative to the order shown. Also, two or more blocks shown in succession may be executed concurrently or with partial concurrence. Certain blocks also may be omitted.

Some of the steps of the method represented by FIG. 4 are similar to or the same as steps found in the previously described methods. Therefore, for the sake of brevity, similar or common steps will not be repeated in detail. Also, features and steps found in the previously described methods may be added to or replace features and steps found in the method represented by FIG. 4, and vice versa.

In the exemplary method represented by FIG. 4, navigation prompts are provided to the user based on the matching of the user's mental state with previously trained mental states that are associated with various landmarks. A cue and a navigation prompt may be associated with each trained mental state, as represented by a training signal. Navigation prompts may be, for example, left and right turns, lane shifts, instructions to go straight, etc. Cues may be the landmarks themselves, such as buildings, specific stores (e.g., a car dealer, a specified fast food restaurant, etc.), street signs, street names, intersections (e.g., the third street on the left past a bank), and so forth. Also, the cue may be something that is associated with a known landmark. For instance, a corporate logo for a retail store may serve as the cue, and when the user is travelling to the destination and sees the store or corresponding logo on a sign, a matching biosignal may be detected. Each cue may invoke a distinguishable mental state that may be used to form a training signal for later matching when the user is driving, walking, riding a bike, etc. When a match is made the corresponding directional prompt may be audibly and/or visually output to the user. Unlike modern GPS guided navigation, the disclosed navigation technique uses a direction and landmark based approach that may be more cognitively natural to the user. For instance, if one were to ask another person for directions, the person would typically provide the directions in the form of turns or other direction prompts that are associated with a series of landmarks and/or street names.

The logical flow for the biosignal navigation function may begin in block 94 where the biosignal application may be launched and the user may select a biosignal navigation function. Then, in block 96, the user may select a training mode or a use mode. If the user selects the training mode, the logical flow may proceed to block 98. In block 98 the biosignal application 12 may prompt the user to mentally concentrate on (e.g., think about) a cue. As indicated, the user may concentrate on the cue by looking at an image or object that represents the cue or the user may concentrate on an established mental impression of the cue. As indicated, the cue in this method may be a landmark that may assist the user reach his or her intended destination. The user may indicate to the electronic device 10 that the user is concentrating on the cue. This may be accomplished, for example, by pressing and holding a specified button from the keypad 18.

In block 100, while the user concentrates on the cue, a biosignal application 12 may capture a training signal (also known as a training vector). The user may concentrate on the cue. Concentrating on the cue may involve concentrating on the user's mental impression of the cue or may involve observing a visual aid. An exemplary visual aid is an image of a corporate logo associated with a landmark. Other visual aids may include photographs or images from a website that provides “street view” images or 3D “earth view” images. The training signal may contain biosignal data from the user that has a correlation to the landmark and, hence, may contain a representation of the mental state of the user while the user thinks about the landmark. In one embodiment, the capture of biosignal data may continue until sufficient data for a reliable training signal is captured. At that time, the user may be informed that the training is complete and may discontinue concentrating on the visual cue.

Next, in block 102, the user may associate a directional prompt with the training signal. The direction prompt may take the form of spoken words or text that is keyed into the electronic device 10. Recordings of the spoken words may be played back during a use phase to direct the user to the destination. Also, text may be converted to speech and output to the user. Text-based directional prompts also may be displayed. In addition, a graphical directional prompt may be selected by the user and associated with the training signal. During use, the selected graphical prompt may be displayed. Also in block 84, the captured training signal and any directional prompt information may be stored, such as in the above-described database.

In block 104, a determination may be made as to whether training signals and directional prompts are stored for all desired directions involved in reaching the desired destination. If additional directions are desired, the logical flow may return to block 98 for additional training. The resulting training signals may be considered to correspond to an ordered list of waypoints with associated directional prompts to guide the user to the intended destination.

Returning to block 96, if the user chooses the use mode, the logical flow may proceed to block 106. In block 106, the user may start to travel to the intended destination while observing landmarks and other possible visual cues. While the user makes these observations, the biosignal application 12 may monitor biosignal data from the biosignal detection headset 52.

In block 110, the biosignal application 12 determines if the biosignal data contains a representation of the user's mental state that matches the user's mental state as represented in one of the training signals. In one embodiment, a simple data or signal matching engine may be employed. In other embodiments, analysis of the monitored biosignal data and the biosignal data in the training signals may be made to detect patterns or nuances in the respective datasets that match one another. The search for a match in block 110 may continue until the biosignal application 12 has sufficiently high confidence that a match is made. If a match is made with a sufficiently high degree of confidence, the directional prompt corresponding to the matched training signal may be output to the user in block 112.

In one embodiment, the electronic device 10 may monitor turns made while travelling the location. Accelerometers may be used for this purpose. If a directional prompt involves a turn, the turn may be detected and the biosignal application 12 may attempt to match the next training signal from the series of waypoints to the monitored biosignal data. In this manner, the matching algorithm may narrow the scope of the matching between currently monitored biosignal data and previously stored training signals.

In another embodiment, voice inputs may be used to enhance performance. For instance, the user may state the name of a waypoint during training. This name may be stored with the other waypoint information. As the user travels to the destination, the user may not only watch for cues, but may speak the name of the waypoints as they are reached. This may assist the biosignal application 12 in advancing through the ordered sequence of waypoints, especially for subtle direction changes and navigation prompts that instruct the user to continue heading straight.

With additional reference to FIG. 5, illustrated are logical operations to implement an exemplary method of constructing a search string for searching the Internet or a searchable database. The exemplary method may be carried out by executing an embodiment of the biosignal application 12, for example. Thus, the flow chart of FIG. 5 may be thought of as depicting steps of a method carried out by the electronic device 10. Although FIG. 5 shows a specific order of executing functional logic blocks, the order of executing the blocks may be changed relative to the order shown. Also, two or more blocks shown in succession may be executed concurrently or with partial concurrence. Certain blocks also may be omitted.

Some of the steps of the method represented by FIG. 5 are similar to or the same as steps found in the previously described methods. Therefore, for the sake of brevity, similar or common steps will not be repeated in detail. Also, features and steps found in the previously described methods may be added to or replace features and steps found in the method represented by FIG. 5, and vice versa.

In the exemplary method represented by FIG. 5, components of a search string are established based on a match between the user's mental state and a previously trained mental state that is associated with searchable information. The search string may be formulated in a contextual manner in that the search string may be established from a combination of voice input (or text input) and biosignal associations.

The logical flow for the biosignal search function may begin in block 114 where the biosignal application 12 may be launched and the user may select a biosignal search function. Then, in block 116, the user may select a training mode or a use mode. If the user selects the training mode, the logical flow may proceed to block 118. In block 118 the biosignal application 12 may prompt the user to mentally concentrate on (e.g., think about) a cue. As indicated, the user may concentrate on the cue by looking at an image or object that represents the cue or the user may concentrate on an established mental impression of the cue. For the biosignal search function, the cue should correspond to something that the user would like to search at some point in the future. For instance, if the use often undertakes searches for the same topic, the cue may relate to that topic. For purposes of an example, the cue in the following description relates to a music artist for which the user undertakes frequent searches.

The user may indicate to the electronic device 10 that the user is concentrating on the cue. This may be accomplished, for example, by pressing and holding a specified button from the keypad 18.

In block 120, while the user concentrates on the cue, a biosignal application 12 may capture a training signal (also known as a training vector). The use may concentrate on the cue. Concentrating on the cue may involve concentrating on the user's mental impression of the cue or may involve observing a visual aid. An exemplary visual aid is an image of album cover art for an album by the artist for which the user would like to establish a training signal. The training signal may contain biosignal data from the user that has a correlation to the cue and, hence, may contain a representation of the mental state of the user while the user thinks about the cue. In one embodiment, the capture of biosignal data may continue until sufficient data for a reliable training signal is captured. At that time, the user may be informed that the training is complete and may discontinue concentrating on the cue.

Next, in block 122, the user may associate a text string with the training signal. The text string may be keyed in by the user or may be spoken and converted to text. The text string may be used in the construction of a future search string. Following the example of music artist, the text string may be the name of the artist related to the cue for which the training signal was established.

In block 124, the training signal and the text string may be stored. In one embodiment, this data may be stored in the above-described database. The user may carry out the training routine more than once to store training signals for plural cues. Following the example of a music artist, the user may store training signals for additional music artists.

Returning to block 116, if the user chooses the use mode, the logical flow may proceed to block 126. The biosignal search function may make use of both biosignal data and voice input from the user. In block 126, the user may be prompted to concentrate on his or her mental impression of a visual cue of interest (or physical representation of the cue if available to the user) and speak a desired search term or other utterance that is related to the cue. In the example of music artists, exemplary spoken search terms may include “concert dates,” “new releases,” “music chart rankings,” names of songs, names of band members, lyrics from a song, and so forth. It is noted that the user may be prompted to concentrate on the cue for a length of time that is longer than it takes the user to speak the search term or other utterance. This is to facilitate biosignal matching.

In block 128, the spoken search term may be converted to text using any appropriate speech to text converter. As will be described below, the converted text may be used as part of a search string.

In block 130, while the user concentrates on the cue, the biosignal application 12 may monitor a biosignal data from the biosignal detection headset 52. In block 132, the biosignal application 12 determines if the biosignal data contains a representation of the user's mental state that matches the user's mental state as represented in one of the training signals. In one embodiment, a simple data or signal matching engine may be employed. In other embodiments, analysis of the monitored biosignal data and the biosignal data in the training signals may be made to detect patterns or nuances in the respective datasets that match one another. The search for a match in block 132 may continue until the biosignal application 12 has sufficiently high confidence that a match is made or until a predetermined maximum capture time has been exceeded. If a match is not made, the user may repeat the attempt to make a match or the logical routine may end.

If a positive determination is made in block 132, the logical flow may proceed to block 134. In block 134, the biosignal application 12 may construct a search string from the converted text and the text string that is stored in association with the matched training signal. In one embodiment, the words from the voice input and words from the text that is associated with the matched training signal are used as search terms. In one embodiment, the words from both sources may be used in combination to generate the search string. In a more tailored approach, the words may be combined using a weighting technique to give more or less preference to words from the converted text relative to the words associated with the matched training signal. The weighting may depend on predetermined preferences. Alternatively, the weighting may depend on a level of confidence in the match between the monitored biosignal data and the training signal. A low degree of confidence may give a higher weight to the converted text, and vice versa.

In another embodiment, a first search string may be constructed from only one of the converted text or the text associated with the matched training signal. Then, the words from the other body of text may be used to construct a second search string that is used for consistency checking of search results based on the first search string.

In still another embodiment, the user may not be prompted to speak or may choose not speak in block 126. In those situations, the search string may be based on the text associated with the matched training signal.

It is possible that the voice input from the user may be spoken in a manner that cannot be deciphered by a speech to text converter (e.g., mumbled) or inaccurate (e.g., lyrics that are not correct). In this case, the spoken component may not generate words for the search string or may not contribute to search performance. However, it is contemplated that the act of speaking during the monitoring of the biosignal data may contribute to establishing a match with a training signal in this exemplary method by focusing the user's state of mind.

It is possible that more than one tentative match may occur. Using the foregoing example, the matching may match the biosignal data with a training signal for a first artist with eight percent confidence and with a training signal for a second artist with fifty percent confidence. In this case, the two matches may be presented to the user for selection of the appropriate match, or search strings for both potential matches may be constructed. In still another approach, the search string may be constructed using a weighted combination of the text associated with both matched training signals in which greater weight is given to the text associated with the match that has a higher level of confidence. In another embodiment, a first search string may be constructed from the text associated with the match that has a higher level of confidence and, then, the text associated with the other match may be used to construct a second search string that is used for consistency checking of search results based on the first search string.

Following block 134, the logical flow may proceed to block 136 where a search is conducted based on the search string. Search results may be displayed to the user.

Although certain embodiments have been shown and described, it is understood that equivalents and modifications falling within the scope of the appended claims will occur to others who are skilled in the art upon the reading and understanding of this specification.

What is claimed is:

1. A method of taking action in response to biosignals detected from a user, comprising:
establishing a training signal containing biosignal data corresponding to a mental state of the user while the user concentrates on a perceptual cue;
associating a user specified action to be carried out by an electronic device with the training signal;
monitoring biosignal data from the user; and
comparing the monitored biosignal data to the training signal and, if a match between the monitored biosignal data and the training signal is determined, commanding the electronic device to carry out the user specified action.

2. The method of claim 1, wherein the perceptual cue includes a visual representation of an item that is physically observed by the user at the time of establishing the training signal.

3. The method of claim 1, wherein the perceptual cue includes a trademark.

4. The method of claim 1, wherein the user specified action relates to one of a calling function, a messaging function, an audiovisual content playback function, a search function, or a navigation function.

5. The method of claim 1, wherein the perceptual cue includes a visual cue associated with a location, and a position of the electronic device is determined at the time of establishing the training signal.

6. The method of claim 5, wherein the user specified action is determining return directions to the position.

7. The method of claim 1, wherein the user specified action is automatically carried out if the match is made with a level of confidence that is above a predetermined threshold.

8. The method of claim 1, wherein the user specified action is established by recording a macro of steps.

9. The method of claim 1, wherein:
establishing the training signal includes establishing an ordered set of training signals, each containing biosignal data corresponding to a mental state of the user while the user concentrates on a perceptual cue;
for each training signal, the associated user specified action is outputting a navigational prompt associated with the training signal;
the monitoring of biosignal data is carried out as the user travels to a destination; and
if a match between the monitored biosignal data and one of the training signals is determined, the commanding of the electronic device includes outputting the corresponding navigational prompt to the user.

10. The method of claim 9, wherein at least one of the perceptual cues is a representation of a landmark at which a navigational prompt is desired.

11. The method of claim 9, wherein at least one of the perceptual cues is a trademark associated with a landmark at which a navigational prompt is desired.

12. The method of claim 9, further comprising detecting turns made by the user and advancing through the ordered set of training signals based on the detected turns.

13. The method of claim 1, wherein the user specified action is constructing a search string using text representing search criteria that is associated with the training signal.

14. The method of claim 13, further comprising receiving voice input from the user.

15. The method of claim 14, further comprising converting the voice input to text.

16. The method of claim 15, wherein the converted text is made part of the search string.

17. The method of claim 13, wherein the voice input is received during the monitoring.

18. A system for taking action in response to biosignals detected from a user, comprising:
a biosignal detection headset configured to detect biosignals from a user that are indicative of a mental state of the user and output corresponding biosignal data; and
an electronic device that includes an interface to receive the biosignal data from the biosignal detection headset and a control circuit configured to:
establish a training signal containing biosignal data corresponding to a mental state of the user while the user concentrates on a perceptual cue;
associate a user specified action to be carried out by the electronic device with the training signal;
monitor biosignal data from the user; and
compare the monitored biosignal data to the training signal and, if a match between the monitored biosignal data and the training signal is determined, command the electronic device to carry out the user specified action.

19. The system of claim 18, wherein:
the control circuit establishes an ordered set of training signals, each containing biosignal data corresponding to a mental state of the user while the user concentrates on a perceptual cue;
for each training signal, the associated user specified action is outputting a navigational prompt associated with the training signal;
biosignal data is monitored as the user travels to a destination; and
if a match between the monitored biosignal data and one of the training signals is determined, the corresponding navigational prompt is output to the user.

20. The system of claim 18, wherein the user specified action is constructing a search string using text representing search criteria that is associated with the training signal.
